(19) United States
(12) Patent Application Publication
(10) Pub. No.: US 2024/0279735 A1
(43) Pub. Date: Aug. 22, 2024

(54) PRIMERS FOR DETECTING TRACE AMOUNT OF RARE SINGLE- NUCLEOTIDE VARIANT AND METHOD FOR SPECIFICALLY AND SENSITIVELY DETECTING TRACE AMOUNT OF RARE SINGLE-NUCLEOTIDE VARIANT BY USING SAME

(71) Applicant: KOREA UNIVERSITY RESEARCH AND BUSINESS FOUNDATION, Seoul (KR)

(72) Inventors: Cheul Hee JUNG, Seoul (KR); Jun Y SHIN, Seoul (KR)

(21) Appl. No.: 17/787,499

(22) PCT Filed: Dec. 17, 2020
(86) PCT No.: PCT/KR2020/018607

§ 371 Date: Jun. 20, 2022

(30) Foreign Application Priority Data
Dec. 20, 2019 (KR) 10-2019-0171577
Dec. 14, 2020 (KR) 10-2020-0174895

(51) Int. Cl. C12Q 1/6876; C12Q 1/686

(52) U.S. Cl. CPC C12Q 1/6876; C12Q 1/686; C12Q 2600/156

(57) ABSTRACT

The present invention relates to a primer set for detecting a trace amount of a rare single-nucleotide variant and a method for specifically and sensitively detecting a trace amount of a rare single-nucleotide variant by using same. Particularly, the present invention relates to: a primer composition for detecting a single-nucleotide variant (SNV), the primer composition comprising a first primer that comprises a nucleotide sequence complementary to a to-be-detected DNA fragment comprising an SNV to be detected, and at the 3′ end of the primer, 2-6 nucleotide sequences that are not complementary to the to-be-detected DNA fragment; and a method for detecting a trace amount of a rare single-nucleotide variant, the method comprising performing a polymerase chain reaction (PCR) using the composition.

### SEQUENCE LISTING

A Sequence Listing accompanies this application and is submitted as a TXT file named “00034seqlstg.txt” which is 2,590 bytes in size and was created on Nov. 8, 2022. The Sequence Listing is electronically submitted via Patent Center and is incorporated by reference in its entirety.

### TECHNICAL FIELD

The present invention relates to a primer for detecting a trace amount of a rare single-nucleotide variant and a method for specifically and sensitively detecting a trace amount of a rare single-nucleotide variant using the same.

### RELATED ART

A single-nucleotide variant (SNV) refers to a mutation having a single nucleotide change and includes single nucleotide polymorphism (SNP) and point mutation.

Single nucleotide variants or differences in DNA or RNA sequences can lead to significant biological consequences, and such single-nucleotide variants can be considered essential biomarkers for biomedical research and clinical applications. It has long been the focus of nucleic acid biotechnology. MALDI mass spectrometry, DNA sequencing, polymerase chain reaction, microarrays, enzyme-assisted methods, and hybridization-based methods have been developed as conventional methods for detecting single-nucleotide variants.

However, these conventionally developed methods have problems with low specificity for detecting a trace amount of SNV and with low detection efficiency due to poor sensitivity and have limitations in that they take a lot of time and high cost.

Meanwhile, due to the development of biotechnology, single-nucleotide variants related to diseases are being discovered. Thus, interest in technologies for diagnosing and treating diseases at an early stage by detecting them quickly and accurately is increasing.

However, among the technologies developed so far, a technology capable of analyzing a trace amount of a rare single-nucleotide variant with high sensitivity and high specificity has not been developed.

### DETAILED DESCRIPTION

### Technical Problem

Accordingly, the present inventors completed embodiments of the present invention by developing a novel primer capable of analyzing a trace amount of a rare single-nucleotide variant with high sensitivity and high specificity, and a novel PCR method capable of detecting a trace amount of a rare single-nucleotide variant using the same.

Accordingly, an object of the present invention is to provide a primer composition for detecting a single-nucleotide variant capable of detecting a trace amount of a rare single-nucleotide variant with high sensitivity and high specificity.

Another object of the present invention is to provide a kit for detecting single-nucleotide variants, the kit including the primer composition for detecting single-nucleotide variants of the present invention.

Another object of the present invention is to provide a method for detecting a trace amount of a rare single-nucleotide variant.

### Solution to the Problem

Accordingly, the present invention provides a primer composition for detecting a single-nucleotide variant, the composition including a first primer that includes a nucleotide sequence complementary to the to-be-detected DNA fragment including a single nucleotide variant to be detected, and 2 to 6 nucleotide sequences that are not complementary to the to-be-detected DNA fragment at the 3′ end of the primer.

In an embodiment of the present invention, the composition may further include a second primer that includes the nucleotide sequence complementary to the to-be-detected DNA fragment, in which the to-be-detected DNA fragment is a wild-type DNA fragment that does not contain a single nucleotide variant, and 2 to 6 nucleotide sequences that are not complementary to the wild-type DNA fragment at the 3′ end of the primer, in which the 3′ end is phosphorylated.

In an embodiment of the present invention, the primer composition for detecting a single-nucleotide variant may be for polymerase chain reaction (PCR).

In addition, the present invention provides a kit for detecting a single nucleotide variant, the kit including the primer composition for detecting a single-nucleotide variant of the present invention.

In addition, the present invention provides a method for detecting a trace amount of a rare single-nucleotide variant, the method including performing a polymerase chain reaction (PCR) using the primer composition for detecting a single-nucleotide variant of the present invention with the to-be-detected DNA fragment including a single nucleotide variant as a template.

In an embodiment of the present invention, the polymerase chain reaction may be performed using a DNA polymerase without 3′->5′ exonuclease activity.

In an embodiment of the present invention, when there is a single-nucleotide variant in the to-be-detected DNA fragment, an amplification product by polymerase chain reaction (PCR) may be generated, and when there is no single-nucleotide variant in the to-be-detected DNA fragment, an amplification product by polymerase chain reaction (PCR) may be not generated.

### Advantageous Effects of Embodiments of the Invention

The present invention relates to a detection primer capable of highly specifically and sensitively detecting a trace amount of a rare single-nucleotide variant, and a method for detecting a rare single-nucleotide variant in a very small amount using the same. The addition of a non-complementary nucleotide at the 3′ end of the primer for detecting single-nucleotide variants according to the present invention has the effect of significantly increasing the detection specificity. In particular, when the primer designed in the present invention is completely sequence-complementarily bound to the single-nucleotide variant, the length of the non-complementary nucleotide at the 3′ end is maintained, whereas when it is bound to the template strand with a single nucleotide mismatch, the length of the non-complementary nucleotide at the 3′ end is increased. Therefore, the primer provided in the present invention has the effect of very specifically amplifying only the single-nucleotide variant (SNV). In addition, when a wild-type primer having a non-complementary nucleotide and a phosphate group at the 3′ end is used together, detection specificity can be further increased. Therefore, the method for detecting a single-nucleotide variant provided by the present invention does not require a sophisticated and complicated primer design so that it is very effective in terms of time, labor, and cost. Accordingly, the primer provided in the present invention can very specifically and sensitively detect a rare single-nucleotide variant in a trace amount.

### BRIEF DESCRIPTION OF THE DRAWINGS

The foregoing and other objects, features, and advantages of the invention, as well as the following detailed description of the embodiments, will be better understood when read in conjunction with the accompanying drawings. For the purpose of illustrating the invention, there is shown in the drawings an exemplary embodiment that is presently preferred, it being understood, however, that the invention is not intended to be limited to the details shown because various modifications and structural changes may be made therein without departing from the spirit of the invention and within the scope and range of equivalents of the claims. The use of the same reference numerals or symbols in different drawings indicates similar or identical items.

FIG. 1 is a schematic view showing a process for detecting a trace amount of a rare single-nucleotide variant by PCR using the primers designed in the present invention.

FIG. 2 is a schematic view showing a process of analyzing the detection sensitivity of a single-nucleotide variant (SNV) by a PCR method using the primers designed in an embodiment of the present invention.

FIG. 3 is a view showing the results of confirming the PCR sensitivity of the MT primer to various copies of the MT Ultramer template synthesized in an embodiment of the present invention by gel electrophoresis.

FIG. 4 is a schematic view showing a process of analyzing the detection specificity of a single-nucleotide variant by a PCR method using the primers designed in an embodiment of the present invention.

FIG. 5 is a schematic view showing a process of analyzing the detection sensitivity and specificity of a single-nucleotide variant for a mixture of various ratios of MT Ultramer template and WT Ultramer template synthesized by adding nucleotide (T) 30 mer in an embodiment of the present invention by a PCR method using the primers designed in the present invention.

FIG. 6 is a view showing the results of analyzing the detection sensitivity and specificity by gel electrophoresis after performing PCR for a mixture of various ratios of MT Ultramer template and WT Ultramer template synthesized by adding nucleotide (T) 30 mer in an embodiment of the present invention using the primers designed in the present invention.

### BEST MODE FOR CARRYING OUT THE IDEA OF THE INVENTION

Embodiments of the present invention provide a primer for detecting a trace amount of a rare single-nucleotide variant and a method for specifically and sensitively detecting a trace amount of a rare single-nucleotide variant using the same.

A single-nucleotide variant related to a disease exists in a trace amount in the body. Thus, it is an important marker for disease diagnosis, but there is a problem in that it cannot be accurately and quickly detected.

Accordingly, the present inventors have studied a method for specifically, sensitively, and rapidly detecting a trace amount of a rare single-nucleotide variant. In the meantime, the present inventors have devised a primer composition for detecting a trace amount of a rare single-nucleotide variant and have identified that it is possible to quickly and accurately detect a rare single-nucleotide variant with high specificity and sensitivity even with a simple analysis method of the polymerase chain reaction.

Therefore, embodiments of the present invention can provide a primer composition for detecting a single-nucleotide variant, in which the composition includes a first primer that includes a nucleotide sequence complementary to the to-be-detected DNA fragment including a single nucleotide variant to be detected, and 2 to 6 nucleotide sequences that are not complementary to the to-be-detected DNA fragment at the 3′ end of the primer.

In addition, the primer composition for detecting a single-nucleotide variant of the present invention may further include a second primer that includes a nucleotide sequence complementary to the to-be-detected DNA fragment, in which the to-be-detected DNA fragment is a wild-type DNA fragment that does not contain a single nucleotide variant, and 2 to 6 nucleotide sequences that are not complementary to the wild-type DNA fragment at the 3′ end of the primer, in which the 3′ end is phosphorylated.

That is, the primer composition for detecting a single nucleotide of the present invention includes an SNV-specific primer (a first primer), in which the first primer is designed to necessarily include a nucleotide sequence complementary to the to-be-detected DNA fragment including a single-nucleotide variant (SNV) and a nucleotide sequence that is not complementary to the to-be-detected DNA fragment at the 3′ end of the first primer. In this case, the non-complementary nucleotide sequence may consist of 2 to 10 nucleotide sequences, preferably 2 to 6 nucleotide sequences, and more preferably 2 to 3 nucleotide sequences.

In addition, the primer composition for detecting a single nucleotide of the present invention may further include a WT-specific primer (a second primer), in which the second primer is designed to necessarily include the nucleotide sequence complementary to the wild-type DNA fragment that does not contain a single nucleotide variant, and nucleotide sequences that are not complementary to the wild-type DNA fragment at the 3′ end of the second primer. In this case, the non-complementary nucleotide sequence may consist of 2 to 10 nucleotide sequences, preferably 2 to 6 nucleotide sequences, and more preferably 2 to 3 nucleotide sequences.

Here, the non-complementary nucleotide sequence that may be included in the first primer and the second primer may be any nucleotide sequence that is non-complementary to the nucleotide sequence of the to-be-detected DNA fragment. In addition, the non-complementary nucleotide sequences of the first primer and the second primer are the same.

In addition, the 3′ end of the second primer is designed to be phosphorylated.

In embodiments of the present invention, the second primer in which the 3′ end is phosphorylated may be used to block the PCR amplification reaction for the wild-type DNA fragment that does not contain the single-nucleotide variant, thereby very specifically and sensitively detecting the target single-nucleotide variant by the first primer.

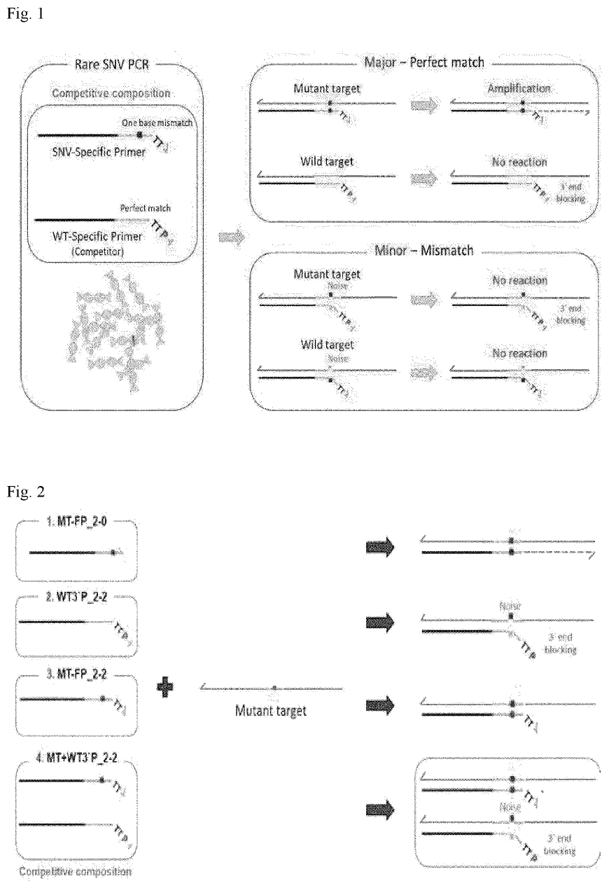

A schematic view of the process of detecting a single-nucleotide variant using the primer composition for detecting a single nucleotide devised in the present invention is shown in FIG. 1. The present invention may perform a polymerase chain reaction (PCR) using a competitive primer composition including a first primer and a second primer in order to very specifically and sensitively detect a trace amount of a single-nucleotide variant (SNV). In this case, the SNV-specific primer has a structure including a non-complementary nucleotide at the 3′ end, and the WT-specific primer has a structure including a non-complementary nucleotide and a phosphate group for amplification blocking at the 3′ end. The difference between the two primers is the single-nucleotide sequence on the 3′ end region (blue region in FIG. 1). These SNV-specific primers and WT-specific primers mainly bind to a perfect match template. Therefore, due to this bonding, the two primers have a structure in which a short single strand exists at the 3′ end due to the non-complementary nucleotide sequence, but the amplification reaction is performed using a DNA polymerase without 3′->5′ exonuclease activity. Therefore, the WT-specific primer cannot be amplified by PCR reaction because phosphoric acid is present at the 3′ end. In addition, the SNV-specific primer and the WT-specific primer can bind to the mismatched template in a very small ratio. As a result, mismatched binding forms noise, and thus the two primers have a relatively long single-stranded structure at the 3′ end. For this reason, the DNA polymerase without 3′->5′ exonuclease activity does not recognize the 3′ end of the primer so that the amplification reaction does not proceed.

Therefore, since the PCR amplification reaction can proceed only when the SNV-specific primer binds to only the SNV template strand, non-specific PCR reaction products are not generated and only a trace amount of the target SNV can be amplified. Thus, the primer composition of the present invention may be used to detect a trace amount of a single-nucleotide variant with high sensitivity and specificity.

In this regard, in one embodiment of the present invention, a G12D mutant DNA fragment of the KRAS gene, known to be highly related to cancer development, was prepared and used as a template for PCR analysis. In this case, a single-stranded template DNA sequence containing the G12D mutation of the KRAS gene is represented by SEQ ID NO: 1, and the single-stranded template DNA sequence including the G12D wild-type sequence of the KRAS gene is represented by SEQ ID NO: 2.

In addition, a reverse primer having a sequence commonly completely complementary to the templates represented by SEQ ID NO: 1 and SEQ ID NO: 2 was designed and represented by SEQ ID NO: 5.

In one embodiment of the present invention, the forward primer sequence represented by SEQ ID NO: 6 including a nucleotide sequence complementary to the template DNA sequence represented by SEQ ID NO: 1, which is a single strand containing the G12D mutation of the RAS gene and two non-complementary nucleotide sequences ‘TT’ at the 3′ end was prepared as the first primer.

In addition, in one embodiment of the present invention, the forward primer sequence represented by SEQ ID NO: 7 including a nucleotide sequence complementary to the template DNA sequence represented by SEQ ID NO: 2, which is a single strand containing the G12D wild-type sequence of the KRAS gene and two non-complementary nucleotide sequences ‘TT’ at the 3′ end was prepared as the second primer, in which the primer was phosphorylated at the 3′ end.

PCR was performed on the first primer and/or the second primer together with the reverse primer represented by SEQ ID NO: 5. As a result, it was found that the G12D single-nucleotide variant of the KRAS gene could be detected with high specificity and sensitivity. Further, it was found that the detection was possible only with the first primer. Further, it was found that the detection was possible with the highest specificity and sensitivity when the first primer and/or the second primer were used together.

Accordingly, the present invention can provide a primer composition for detecting a single-nucleotide variant, and a kit for detecting a single-nucleotide variant including the primer for detecting a single-nucleotide variant.

In the present invention, the term ‘primer’ refers to a short single-stranded oligonucleotide that serves as a starting point for DNA synthesis. A primer binds specifically to a polynucleotide as a template under suitable buffer and temperature conditions, and DNA polymerase adds a nucleoside triphosphate having a nucleotide complementary to that of the template DNA to the primer to link it so that DNA is synthesized. The temperature (melting temperature, Tm) at which the primer binds to the template strand may vary depending on the composition and length of the nucleotide.

In addition, the sequence of the primer does not need to have a sequence that is completely complementary to a partial nucleotide sequence of the template, and it is sufficient as long as it has sufficient complementarity within a range capable of hybridizing with the template to perform the intrinsic function of the primer.

In addition, the kit for detecting the single-nucleotide variant of the present invention may include the above-described primer composition for detecting the single-nucleotide variant of the present invention, and the kit may include a test tube or other suitable container, reaction buffer, dNTPs, DNA polymerase and distilled water, and the like.

Furthermore, the present invention can provide a method for detecting a trace amount of a rare single-nucleotide variant (SNV), in which the method includes a step of performing a polymerase chain reaction (PCR) using a to-be-detected DNA fragment including a single-nucleotide variant (SNV) as a template using the primer composition of the present invention.

In this case, the polymerase chain reaction (PCR) is performed using a DNA polymerase without 3′->5′ exonuclease activity.

In addition, in the case of the method of the present invention, when a single-nucleotide variant exists in the to-be-detected DNA fragment, an amplification product is generated by polymerase chain reaction (PCR). When a single-nucleotide variant does not exist in the to-be-detected DNA fragment, an amplification product is not generated by polymerase chain reaction (PCR).

Therefore, embodiments of the method of the present invention has the effect of detecting with high sensitivity and specificity only the to-be-detected DNA in which the desired single-nucleotide variant exists so that the single-nucleotide variant present in a trace amount related to the disease can be easily detected and analyzed.

### MODE(S) FOR CARRYING OUT THE INVENTION

Hereinafter, the present invention will be described in more detail through examples. These examples are for explaining the present invention in more detail, and the scope of the present invention is not limited to these examples.

### Experiment Method and Preparation Example

All synthesized nucleotides used in the examples of the present invention were ordered from Integrated DNA Technologies, Inc. (IDT) and used. The synthesized nucleotide was received in a freeze-dried state, diluted with TE buffer (10 mM Tris-HCl, 0.1 mM EDTA, pH 8.0), and cultured in a mixing block (MB102; Bioer) at 50° C. for 1 hour. Then, the nucleotide was used in the experiment. In addition, the synthesized nucleotides used in the real-time PCR of the present invention are shown in Table 1 below.

TABLE 1

| | | | | SEQ |
|---|---|---|---|---|
| | | Sequence | Length | ID |
| | Name | (5′ to 3′) | (bp) | NO: |
| 1-1 | Mutant | [symbol] | 195 | 1 |
| | type | | | |
| | Ultramer | | | |
| | (template) | | | |
| 1-2 | Wild type | [symbol] | 195 | 2 |
| | Ultramer | | | |
| | (template) | | | |
| 2 | Mutant | [symbol] | 195 | 3 |
| | type | | | |
| | T30 | | | |
| | Ultramer | | | |
| | (template) | | | |
| 3 | Forward | [symbol] | [symbol] | 4 |
| | Primer | | | |
| | [symbol] | | | |
| 4 | Reverse | [symbol] | [symbol] | 5 |
| | Primer | | | |
| 5 | Mutant- | [symbol] | [symbol] | 6 |
| | specific | | | |
| | Forward | | | |
| | Primer | | | |
| 6 | Wild- | | [symbol] | 7 |
| | specific | | | |
| | Forward | | | |
| | Primer | | | |
| 7 | Taqman | [symbol] | [symbol] | |
| | Probe | | | |

[symbol]

indicates data missing or illegible when filed

In Table 1, templates 1-1 & 1-2 consist of a single-stranded Ultramer (195 nt) containing the KRAS gene G12D Mutant Type sequence and a single-stranded Ultramer (195 nt) containing the KRAS gene G12D Wild Type sequence, respectively and each single stranded Ultramer has a phosphorylation modification at the 3′ end.

In Table 1, the Mutant Type T30 Ultramer Template consists of a single-stranded Ultramer (195 nt) containing the KRAS gene G12D Mutant Type sequence and nucleotide T 30 mer and has a phosphorylation modification at the 3′ end.

In Table 1, Forward Primer (FP) has a completely complementary sequence to the KRAS gene G12D Mutant Type Ultramer (template).

Reverse Primer (RP) has a commonly completely complementary sequence to KRAS gene G12D Mutant Type and KRAS gene G12D Wild Type Ultramer (template).

In Table 1, Mutant-specific Forward Primer (FP) has a sequence complementary to only the KRAS gene G12D Mutant Type template and has non-complementary nucleotides in the 3′ end region.

In Table 1, Wild-specific Forward Primer (FP) has a sequence complementary to only the KRAS gene G12D wild-type template and has non-complementary nucleotides in the 3′ end region. In addition, phosphorylation modification is made at the 3′ end.

In addition, Taqman Probe was used for the signal detection method of real-time PCR amplification products. Real-time PCR was performed using the StepOnePlus Real-Time PCR System (Applied Biosystems) equipment, and the AmpliTaq Gold™ 360 Master Mix (Applied Biosystems™) was used as the DNA polymerase used for real-time PCR.

In addition, a product of Thermo Fisher Scientific was used for electrophoresis in the experiment of the present invention. Otherwise, the product performed in each example was separately indicated.

For polyacrylamide gel electrophoresis (PAGE), 2 μl of Novex TBE-Sample Buffer (5×) was added to each sample, and they were well mixed. 10 ul of the mixture was dispensed on Invitrogen 10% TBE gel, and electrophoresis was performed at 180 V for 35 minutes. After electrophoresis, the gel was well separated, diluted with 5 ul of SYBR Gold Nucleic Acid gel stain in 100 ml of distilled water, and then they were reacted in ROCKER (RF200; BLE) for 15 minutes.

### Example 1

### SNV-PCR Performance and Sensitivity Confirmation Using Primer Set According to Present Invention

In the preparation example, the SNV detection sensitivity for the primers derived in the present invention was analyzed by measuring the Ct value using the template DNA and each primer in Table 1 designed by the present inventors. Specifically, real-time PCR was performed for a Mutant Type Ultramer (template) using a Mutant-specific Forward primer and a Wild-specific Forward primer (non-complementary nucleotides: 0, 2 nt). The conditions of real-time PCR and the composition for each experimental group are described in Table 2 below. The real-time PCR conditions are described in Table 3 below. Each experimental group consists of samples according to the template concentration. A schematic diagram of SNV-PCR performance for sensitivity analysis according to the present invention described above is shown in FIG. 2.

TABLE 2

| | Primer | Mark | Reactants |
|---|---|---|---|
| Ex. | Mutant-specific | MT- [symbol] P_2-0 | Mutant type Ultramer template (100 [symbol] -100 |
| Group 1 | Forward Primer | | nM) 2.5 [symbol] 2X [symbol] Gold 360 Master |
| | (Non-Complementary | | Mix 12.5 [symbol] 10 [symbol] Reverse Primer 0.5 [symbol] |
| | nucleotides: 0 nt) | | 10 [symbol] Mutant-specific Forward Primer (Non- |
| | | | Complementary nucleotides: 0 nt) 0.5 [symbol] 5 [symbol] |
| | | | [symbol] Probe 0.5 [symbol] Nuclease-Free Water 8.5 [symbol] |
| Ex. | Wild-specific | WT3′P-FP_2-2 | Mutant type Ultramer template (100 [symbol] -100 |
| Group 2 | Forward Primer | | nM) 2.5 [symbol] 2X [symbol] Gold 360 Master |
| | (Non-Complementary | | Mix 12.5 [symbol] 10 [symbol] Reverse Primer 0.5 [symbol] |
| | nucleotides: 2 nt) | | 10 [symbol] Wild-specific Forward Primer |
| | | | Complementary nucleotides: 2 nt 0.5 [symbol] 5 [symbol] |
| | | | Probe 0.5 [symbol] Nuclease-Free Water 8.5 [symbol] |
| Ex. | Mutant-specific | MT-FP_ [symbol] -2 | Mutant type Ultramer template (100 [symbol] -100 |
| Group 3 | Forward Primer | | nM) 2.5 [symbol] 2X [symbol] Gold 360 Master |
| | (Non-Complementary | | Mix 12.5 [symbol] 10 [symbol] Reverse Primer 0.5 [symbol] |
| | nucleotides: 2 nt) | | 10 [symbol] Mutant-specific Forward Primer (Non- |
| | | | Complementary nucleotides: 2 nt) 0.5 [symbol] 5 [symbol] |
| | | | [symbol] Probe 0.5 [symbol] Nuclease-Free Water |
| | | | 8.5 [symbol] |
| Ex. | Mutant-specific | MT + | Mutant type Ultramer template (100 [symbol] -100 |
| Group 4 | Forward Primer + | WT3′P_2-2 | nM) 2.5 [symbol] 2X [symbol] Gold 360 Master |
| | Wild-specific | | Mix 12.5 [symbol] 10 [symbol] Reverse Primer 0.5 [symbol] |
| | Forward Primer | | 10 [symbol] Mutant-specific Forward Primer (Non- |
| | (Non-Complementary | | Complementary nucleotides: 2 nt 0.5 [symbol] 10 [symbol] |
| | nucleotides: 2 nt) | | Wild-specific Forward Primer ( [symbol] |
| | | | nucleotides: 2 nt) 0.5 [symbol] 5 [symbol] M [symbol] |
| | | | Probe 0.5 [symbol] Nuclease-Free Water 8 [symbol] |

[symbol]

indicates data missing or illegible when filed

TABLE 3

| | | |
|---|---|---|
| Step | Temp | Time |

| Initial Denaturation | | | 95° C. | 10 | min |
|---|---|---|---|---|---|
| | 70 Cycles | Denaturing | 95° C. | 30 | sec |

| Annealing | 56° C. | 1~4 | |
|---|---|---|---|
| | | Ex. Group | |
| | | 30 | sec |
| Extending | 72° C. | 15 | sec |

| | |
|---|---|
| Hold | 4° C. |

The results of the experiment are shown in Table 4 and FIG. 2 below. These results are obtained by confirming the PCR sensitivity of the MT primer to various copies of the synthesized Mutant Type (MT) Ultramer template through real-time PCR. As a result of the analysis, it was confirmed that experimental group 4, that is, the group using the Mutant-specific Forward Primer and Wild-specific Forward Primer (non-complementary nucleotide: 2 nt) set, was able to detect the SNV of KRAS gene G12D with high sensitivity compared to other experimental groups. Specifically, it was shown to have PCR sensitivity of MT template 10² copy (Ct value. 59) in less than 60 cycles of PCR.

TABLE 4
Real time PCR - Sensitivity (Ct value)

MT template Conc.

| | 1. 100 [symbol] | 2. 10 [symbol] | 3. 1 [symbol] | 4. 100 [symbol] |
|---|---|---|---|---|
| Copy number | 10 [symbol] | 10 [symbol] | 10 [symbol] | 10 [symbol] |
| 1. MT-FP_2-0 | 25.7 | 28.1 | 33 | 37.6 |
| 2. WT3′P_2-2 | — | — | — | — |
| 3. MT-FP_2-2 | 39.9 | 44.9 | 52 | 51.2 |
| 4. MT + WT3′P_2-2 | 41.5 | 49 | 54.1 | 59 |

[symbol]

indicates data missing or illegible when filed

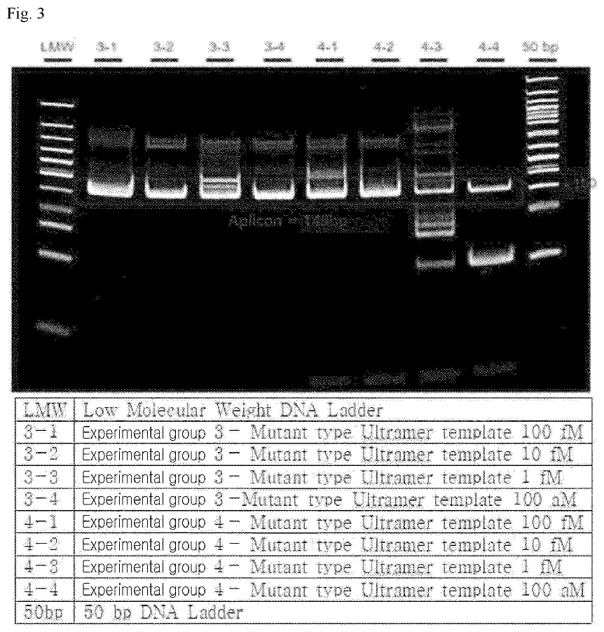

In addition, PCR sensitivity was also confirmed through gel electrophoresis using each of the primers of Experimental Group 3 and Experimental Group 4 of Table 3 for various copies of the synthesized Mutant Type Ultramer template. As a result, it was confirmed to show PCR sensitivity of Mutant Type Ultramer template 10² copy (Ct value. 59) as shown in FIG. 3.

### Example 2

### SNV-PCR Performance and Specificity Confirmation Using Primer Set According to Present Invention

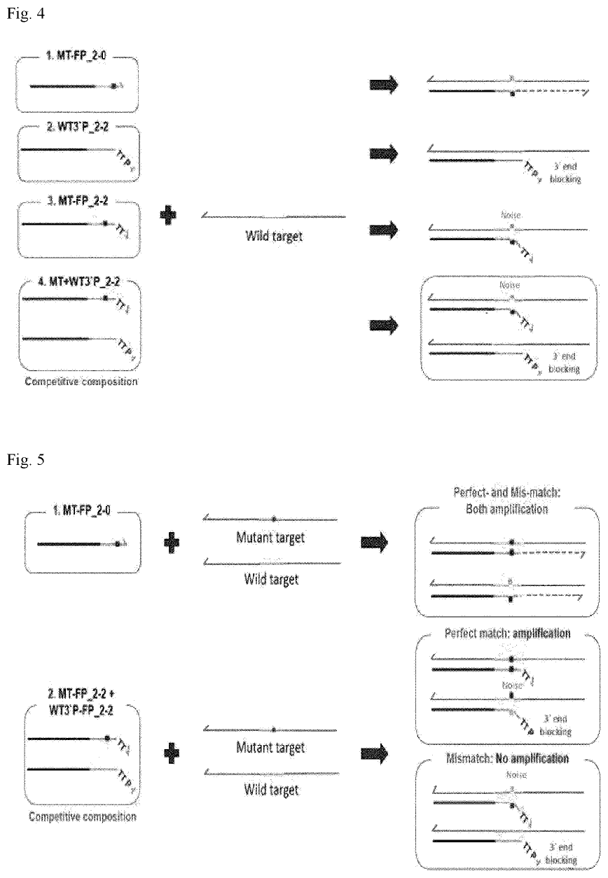

Furthermore, the present inventors analyzed specificity for SNV detection using the primer set designed in the present invention. For this purpose, real-time PCR was performed for the wild-type Ultramer template using Mutant-specific Forward Primer and Wild-specific Forward Primer (non-complementary nucleotide: 0, 2 nt). The real-time PCR condition and the composition of each experimental group are described in Table 5 below. The real-time PCR conditions are described in Table 6 below. Each experimental group consists of a sample according to the template concentration. FIG. 4 illustrates a schematic diagram of performing SNV-PCR for sensitivity analysis according to the present invention described above.

TABLE 5

| | Primer | Mark | Reactants |
|---|---|---|---|
| Ex. | Mutant-specific | MT- [symbol] P_2-0 | Mutant type Ultramer template (100 [symbol] -100 |
| Group 1 | Forward Primer | | nM) 2.5 [symbol] 2X [symbol] Gold 360 Master |
| | (Non-Complementary | | Mix 12.5 [symbol] 10 [symbol] Reverse Primer 0.5 [symbol] |
| | nucleotides: 0 nt) | | 10 [symbol] Mutant-specific Forward Primer (Non- |
| | | | Complementary nucleotides [symbol] 0 nt) 0.5 [symbol] 5 [symbol] |
| | | | [symbol] Probe 0.5 [symbol] Nuclease-Free Water 8.5 [symbol] |
| Ex. | Wild-specific | WT3′P-FP_2-2 | Mutant type Ultramer template (100 [symbol] -100 |
| Group 2 | Forward Primer | | nM) 2.5 [symbol] 2X [symbol] Gold 360 Master |
| | (Non-Complementary | | Mix 12.5 [symbol] 10 [symbol] Reverse Primer 0.5 [symbol] |
| | nucleotides: 2 nt) | | 10 [symbol] Wild-specific Forward Primer |
| | | | Complementary nucleotides: 2 nt 0.5 [symbol] 5 [symbol] |
| | | | Probe 0.5 [symbol] Nuclease-Free Water 8.5 [symbol] |
| Ex. | Mutant-specific | MT-FP_ [symbol] -2 | Mutant type Ultramer template (100 [symbol] -100 |
| Group 3 | Forward Primer | | nM) 2.5 [symbol] 2X [symbol] Gold 360 Master |
| | (Non-Complementary | | Mix 12.5 [symbol] 10 uM Reverse Primer 0.5 [symbol] |
| | nucleotides: 2 nt) | | 10 [symbol] Mutant-specific Forward Primer (Non- |
| | | | Complementary nucleotides: 2 nt) 0.5 [symbol] 5 [symbol] |
| | | | [symbol] Probe 0.5 [symbol] Nuclease-Free Water |
| | | | 8.5 [symbol] |
| Ex. | Mutant-specific | MT + | Mutant type Ultramer template (100 [symbol] -100 |
| Group 4 | Forward Primer + | WT3′P_2-2 | nM) 2.5 [symbol] 2X [symbol] Gold 360 Master |
| | Wild-specific | | Mix 12.5 [symbol] 10 [symbol] Reverse Primer 0.5 [symbol] |
| | Forward Primer | | 10 uM Mutant-specific Forward Primer (Non- |
| | (Non-Complementary | | Complementary [symbol] 2 nt 0.5 [symbol] 10 [symbol] |
| | nucleotides: 2 nt) | | Wild-specific Forward Primer ( [symbol] |
| | | | nucleotides: 2 nt) 0.5 [symbol] 5 [symbol] M [symbol] |
| | | | Probe 0.5 [symbol] Nuclease-Free Water 8 [symbol] |

[symbol]

indicates data missing or illegible when filed

TABLE 6

| | | |
|---|---|---|
| Step | Temp | Time |

| Initial Denaturation | | | 95° C. | 10 | min |
|---|---|---|---|---|---|
| | 70 Cycles | Denaturing | 95° C. | 30 | sec |

| Annealing | 56° C. | 1~4 | |
|---|---|---|---|
| | | Ex. Group | |
| | | 30 | sec |
| Extending | 72° C. | 15 | sec |

| | | |
|---|---|---|
| Hold | 4° C. | — |

As a result of SNV detection specificity analysis of the primer set designed in the present invention, as shown in Table 7 below: experimental group 4, that is, Mutant-specific Forward Primer+Wild-specific Forward Primer (non-complementary nucleotide: 2 nt) set-used group was able to detect SNV of KRAS gene G12D with higher specificity compared to other experimental groups. Considering the PCR sensitivity of MT template 10² copy (Ct value. 59) in less than 60 cycles of PCR, these results showed 10³-fold specificity in PCR 60 cycles or more, and 10⁴-fold specificity in less than 60 PCR cycles.

TABLE 7
Real-time PCR - Specificity (Ct value)

| | WT template Conc. | | | | | |
|---|---|---|---|---|---|---|
| | 1.10 pM | 2.1 pM | 3.100 fM | 4.10 fM | 5.1 fM | 6.100 aM |
| | Copy number | | | | | |
| | 10⁷ | 10⁶ | 10⁵ | 10⁴ | 10³ | 10² |
| 1. MT_2-0 | 17.8 | 21.5 | 26.4 | 30.1 | 33.9 | 37.7 |
| 2. WT3′P_2-2 | — | — | — | — | — | — |
| 3. MT_2-2 | 42.2 | 42.3 | 49.6 | — | — | — |
| 4. MT + WT3 [symbol] P_2-2 | 42.8 | 47.5 | 60.7 | — | — | — |

[symbol]

indicates data missing or illegible when filed

Therefore, these results indicated that when the primer set designed in the present invention is used, rare SNVs, especially SNVs in a trace amount, can be quickly and accurately detected with excellent sensitivity and specificity through the simple method of PCR.

### Example 3

### Confirmation of Sensitivity and Specificity According to SNV-PCR Using Primer Set According to Present Invention

In addition, the present inventors analyzed the sensitivity and specificity for SNV detection using the primers designed in the present invention for the mixed template DNA in which the Mutant Type T30 Ultramer template and Wild Type Ultramer template were mixed. For this purpose, real-time PCR was performed by using a mixture of templates (Mutant Type T30 Ultramer template+Wild Type Ultramer template mixture) in various ratios and Mutant-specific Forward Primer and Wild-specific Forward Primer (non-complementary nucleotides: 0, 2 nt). The composition for each experimental group is described in Table 8 below. The real-time PCR conditions are shown in Table 9 below. FIG. 5 illustrates a schematic diagram of the RT-PCR process for sensitivity and specificity analysis according to the present invention described above.

In addition, a Mutant Type T30 Ultramer template was used in this example. When two types of Ultramer templates (MT/WT) are used together in a real-time PCR reaction, the TaqMan probe is commonly annealed to both the PCR product by MT Ultramer and the PCR product by WT Ultramer, thereby generating a signal. Therefore, for more accurate amplification confirmation, the difference in length was made so that it could be easily confirmed through gel electrophoresis, whether amplified by the MT Ultramer template or the WT Ultramer template.

TABLE 8

| | Primer | Mark | Reactants |
|---|---|---|---|
| Ex. | Mutant-specific | MT-FP_2-0 | Mutant Type T30 Ultramer (100 nM) |
| Group 1 | Forward Primer | | 2.5 [symbol] wild type Ultramer template (1 |
| | (Non-Complementary | | [symbol] -100 nM) 2.5 [symbol] 2X [symbol] Gold |
| | nucleotides: 0 nt) | | 360 Master Mix 12.5 [symbol] 10 [symbol] |
| | | | Reverse Primer 0.5 [symbol] 10 [symbol] |
| | | | Mutant-specific Froward Primer (Non- |
| | | | complementary nucleotide: 0 nt) 0.5 [symbol] 5 [symbol] |
| | | | [symbol] Probe 0.5 [symbol] Nuclease-Free |
| | | | Water [symbol] |
| Ex. | Mutant-specific | MT-FP_2-2 + | Mutant Type T30 Ultramer (100 nM) |
| Group 2 | Forward Primer + | WT3′P-FP_2-2 | 2.5 [symbol] wild type Ultramer template (1 |
| | Wild-specific | | [symbol] -100 nM) 2.5 [symbol] 2X [symbol] Gold |
| | Forward Primer | | 360 Master Mix 12.5 [symbol] 10 [symbol] |
| | (Non-Complementary | | Reverse Primer 0.5 [symbol] 10 [symbol] |
| | nucleotides: 2 nt) | | Mutant-specific Forward Primer (Non- |
| | | | complementary nucleotide: 2 nt) 0.5 [symbol] 10 [symbol] |
| | | | Wild-specific Forward Primer (Non- |
| | | | complementary nucleotide: 2 nt) 0.5 [symbol] 5 [symbol] |
| | | | [symbol] Probe 0.5 [symbol] Nuclease-Free |
| | | | Water 5.5 [symbol] |

[symbol]

indicates data missing or illegible when filed

TABLE 9

| | | |
|---|---|---|
| Step | Temp | Time |

| Initial Denaturation | | | 95° C. | 10 | min |
|---|---|---|---|---|---|
| | 70 Cycles | Denaturing | 95° C. | 30 | sec |

| Annealing | 56° C. | 1, 2 | |
|---|---|---|---|
| | | Ex. Group | |
| | | 30 | sec |
| Extending | 72° C. | 15 | sec |

| | | |
|---|---|---|
| Hold | 4° C. | — |

In order to make a difference in amplicon size, sensitivity and specificity through real-time PCR analysis using MT primers for a mixture of MT T30 Ultramer template and WT Ultramer template in various ratios, which are additionally synthesized with nucleotide (T) 30 mer was confirmed through Ct value analysis and also analyzed through gel electrophoresis.

TABLE 10
Real-time PCR - Sensitivity and Specificity (Ct value)

| | MT/WT template Conc. | | | | | |
|---|---|---|---|---|---|---|
| | 1.100 aM/0 | 2.100 aM/1 pM | 3.100 aM/100 fM | 4.100 aM/10 fM | 5.100 aM/1 fM | 6.100 aM/100 aM |
| | MT/WT copy number | | | | | |
| | 10 [symbol] /0 | 10 [symbol] /10 [symbol] | 10 [symbol] /10 [symbol] | 10 [symbol] /10 [symbol] | 10 [symbol] /10 [symbol] | 10 [symbol] /10 [symbol] |
| | Variant allele frequency (VAF) | | | | | |
| | 100% | 0.01% | 0.1% | 1% | 10% | 50% |
| 1. MT-FP_2-0 | 35.9 | 22 | 25.9 | 32.5 | 35.2 | 35.5 |
| 2. MT-FP_2-2 + | [symbol] | 47.2 | 55.2 | [symbol] | [symbol] | [symbol] |
| WT3 [symbol] P-PP_2-2 | | | | | | |

[symbol]

indicates data missing or illegible when filed

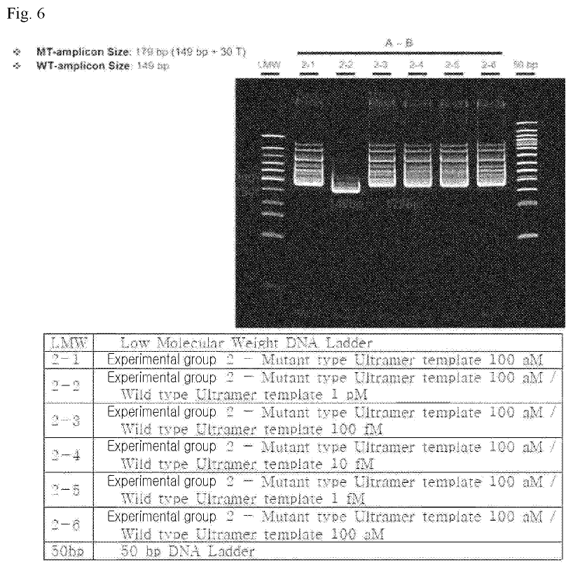

As a result, as shown in Table 10 and FIG. 6, even in the result of using a mixed template of MT and WT using the primer of the present invention, it was confirmed that it showed PCR sensitivity of perfect-matched MT template 10² copy regardless of the (mismatched) WT template copy and exhibited 10³-fold PCR specificity (VAF. 0.1%).

Therefore, from these results, the present inventors found that, when using the primers designed in the present invention, a trace amount of SNV can be detected with high sensitivity and specificity by a simple method of PCR.

The present invention has been described focusing on preferred embodiments thereof. Those of ordinary skill in the art to which the present invention pertains will understand that the present invention can be implemented in a modified form without departing from the essential characteristics of the present invention. Therefore, the disclosed examples are to be considered in an illustrative rather than a restrictive view. The scope of the present invention is indicated in the claims rather than the foregoing description, and all differences within the scope equivalent thereto should be construed as being included in the present invention.

Many modifications to the above embodiments may be made without altering the nature of the invention. The dimensions and shapes of the components and the construction materials may be modified for particular circumstances. While various embodiments have been described above, it should be understood that they have been presented by way of example only, and not as limitations.

1. A primer composition for detecting a single-nucleotide variant (SNV), the composition comprising a first primer that includes a nucleotide sequence complementary to a to-be-detected DNA fragment including a single nucleotide variant to be detected, and 2 to 6 nucleotide sequences that are not complementary to the to-be-detected DNA fragment at a 3′ end of the primer.

2. The primer composition of claim 1, the composition further comprising a second primer that includes the nucleotide sequence complementary to the to-be-detected DNA fragment, wherein the to-be-detected DNA fragment is a wild-type DNA fragment that does not contain a single nucleotide variant, and 2 to 6 nucleotide sequences that are not complementary to the wild-type DNA fragment at the 3′ end of the primer, in which the 3′ end is phosphorylated.

3. The primer composition of claim 1, wherein the composition is for polymerase chain reaction (PCR).

4. A kit for detecting a single nucleotide variant, the kit comprising the composition of claim 1.

5. A method for detecting a trace amount of a rare single-nucleotide variant, the method comprising performing a polymerase chain reaction using the primer composition of claim 1 with a to-be-detected DNA fragment including a single nucleotide variant as a template.

6. The method of claim 5, wherein the polymerase chain reaction (PCR) is performed using a DNA polymerase without 3′->5′ exonuclease activity.

7. The method of claim 5, wherein when there is a single-nucleotide variant in the to-be-detected DNA fragment, an amplification product by the polymerase chain reaction (PCR) is generated, and when there is no single-nucleotide variant in the to-be-detected DNA fragment, the amplification product by polymerase chain reaction (PCR) is not generated.


SEQUENCE LISTING (7 sequences)

SEQ ID NO: 1 - DNA, 195 bp, Artificial Sequence
  Synthetic- Mutant type Ultramer template sequence
aaggcctgctgaaaatgactgaatataaacttgtggtagttggagctgatggcgtaggcaagagtgccttgacgatacagctaattcagaatcattttgtggacgaatatgatccaacaatagaggtaaatcttgttttaatatgcatattactggtgcaggaccattctttgatacagataaaggtttctctga

SEQ ID NO: 2 - DNA, 195 bp, Artificial Sequence
  Synthetic- Wild type Ultramer template sequence
aaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctga

SEQ ID NO: 3 - DNA, 195 bp, Artificial Sequence
  Synthetic- Mutant typeT30 Ultramer template sequence
aatataaacttgtggtagttggagctgatggcgtaggcaagagtgccttgacgatacagctaattcagaatcattttgtggacgaatatgatccaacaatagaggtattttttttttttttttttttttttttttttaatcttgttttaatatgcatattactggtgcaggaccattctttgatacagataaagg

SEQ ID NO: 4 - DNA, 20 bp, Artificial Sequence
  Synthetic- Forward Primer sequence
ttgtggtagttggagctgat

SEQ ID NO: 5 - DNA, 24 bp, Artificial Sequence
  Synthetic- Reverse Primer sequence
ctgtatcaaagaatggtcctgcac

SEQ ID NO: 6 - DNA, 22 bp, Artificial Sequence
  Synthetic- Mutant-specific Forward Primer(2nt)
ttgtggtagttggagctgattt

SEQ ID NO: 7 - DNA, 22 bp, Artificial Sequence
  Synthetic- Wild-specific Forward Primer(2nt)
ttgtggtagttggagctggttt